(19) United States
(12) Patent Application Publication
(10) Pub. No.: US 2015/0223898 A1
(43) Pub. Date: Aug. 13, 2015

(54) FAST LOCKING DEVICE FOR AT LEAST ONE TOOL ON AT LEAST ONE GUIDE ELEMENT

(71) Applicant: GZ MEDITEC S.R.L., Rovereto (TN) (IT)

(72) Inventors: Paolo Merlo, Rovereto (TN) (IT); Christian Rossi, Rovereto (TN) (IT)

(21) Appl. No.: 14/430,119

(22) PCT Filed: Sep. 19, 2013
(86) PCT No.: PCT/IB2013/002055

§ 371 Date: Mar. 20, 2015

(30) Foreign Application Priority Data
Sep. 21, 2012 (IT) BO2012A000509

(51) Int. Cl. A61B 19/00; F16M 13/02

(52) U.S. Cl. CPC A61B 19/26; F16M 13/022; A61B 2019/268

(57) ABSTRACT

A fast locking device for at least one tool (9) on at least a guide element (8) comprising:
a fixing element (2) provided with at least one first opening (29) for the through-passage of at least one tool (9);
a base (3) removably lockable to the fixing element (2), via clutch means, provided with a stop surface (31), opposite to the fixing element (2), at an end of which there is a locking element (38) to at least partially engage the guide element (8);
a hook-like element (4) revolvingly locked to the base (3) between a release condition (A) and a locking condition (B) for the guide element (8);
a cursor element (7) sliding internally to the fixing element (2) and to the base (3) and provided with at least one second opening (79) and one hollow (74) which engages a projection (47) present on the hook-like element (4);
a locking means (6) which internally engages the cursor element (7) and is provided with an inner end (69) that leads to the inside of the first opening (29) and of the second opening (79) and abuts the tool (9);
at least one elastic element (39) interposed between the base (3) and the fixing element (2) which counters their mutual approach.

### DESCRIPTION OF THE INVENTION

The present invention relates to the sector concerning tools for operating theatres and in particular it refers to a fast locking device for at least one tool on at least one guide element.

In the sector of tools for operating theatres, devices are known to fix tools and/or instruments to special guides locked to the operating table in correspondence with the edges or appendices of the operating plane. Such guides feature a rectangular transversal section and dimensions determined by international regulations to standardize the types of locking devices connectable thereto.

The simplest locking devices known so far comprise a pair of reciprocally and integrally locked clamps, the former of which is designed for being fixed to a guide of the operating table and the second is suitable for locking a tool. These clamps are usually driven by separate adjustment elements, for instance consisting of their respective “T”-head screws and engaging appropriate nut screws of the clamp. The two clamps are realized in one body, consequently they feature a fixed angle between the planes of action of the two clamps.

An evolution of said clamps known so far concerns the possibility of modifying the angle between the planes of action of the two clamps, in that the latter are implemented by two separate elements and are coupled to each other by a revolving and removable locking element which allows a mutual rotation of the two clamps as well as the possibility of fixing them in the desired position. The revolving locking element consists of a revolving pin and a third screw which, coupled with a nut screw present on at least one of the two clamps, acts onto the pin to prevent its rotation in the fixed condition.

A further evolution of the devices known so far concerns the replacement of the screw locking elements with snap-in and/or spring locking elements or a combination of the different types mentioned above.

A drawback of the fast locking systems for tools known so far concerns the presence of many locking elements to adjust and/or lock each individual movement of the tool or of the locking device on the guide of the operating table, which often requires the use of both operator's hands to perform the operation.

A further drawback concerns the high number of movable, hence subjected to wear and tear, elements and possible malfunctions present in the devices known so far which render the implementation and the subsequent use of the locking device complex and expensive.

The main purpose of the present invention is to provide a fast locking device for at least one tool that allows to fix said device onto a support guide, to adjust the position of the device and of the tool locked thereto by operating with one hand onto one adjustment element so as to make its use simpler.

A further purpose is to provide a device that comprises a limited number of movable elements in order to reduce both the implementation costs and the maintenance costs as well as to reduce the possible malfunctions.

Another purpose is to provide a device that offers more possibilities of use and adjustments with respect to the locking devices known so far while retaining a structure that is compact and does not hamper any operations performed around there.

The characteristics of the invention are highlighted below with a special reference to the attached drawings, in which:

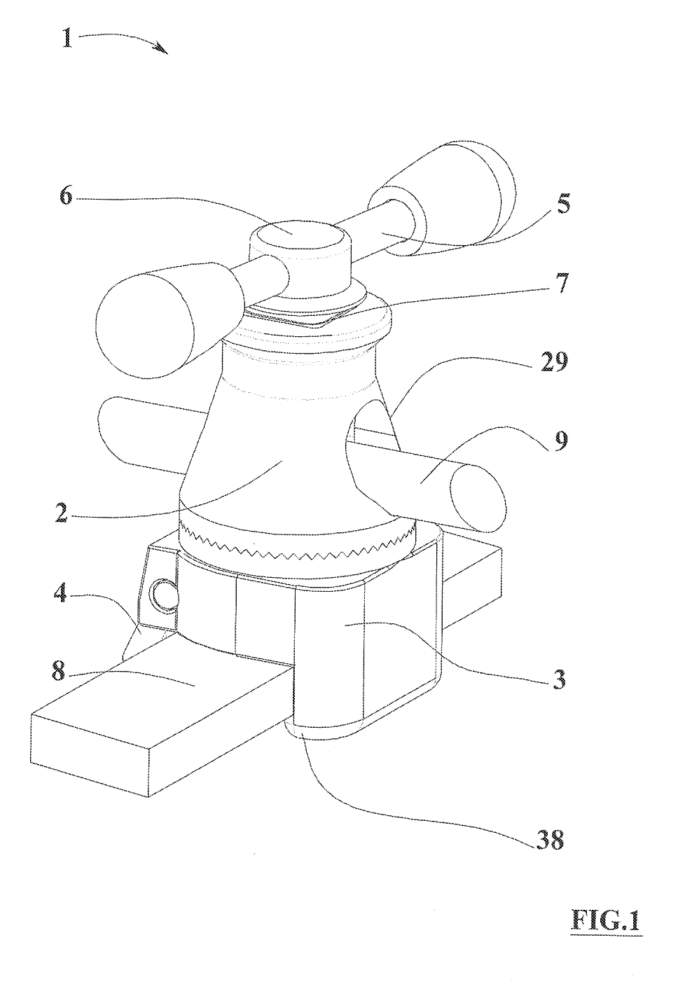

FIG. 1 shows an axonometric view of the fast locking device for tools according to the present invention in an operating condition whereby a tool is fixed to a guide;

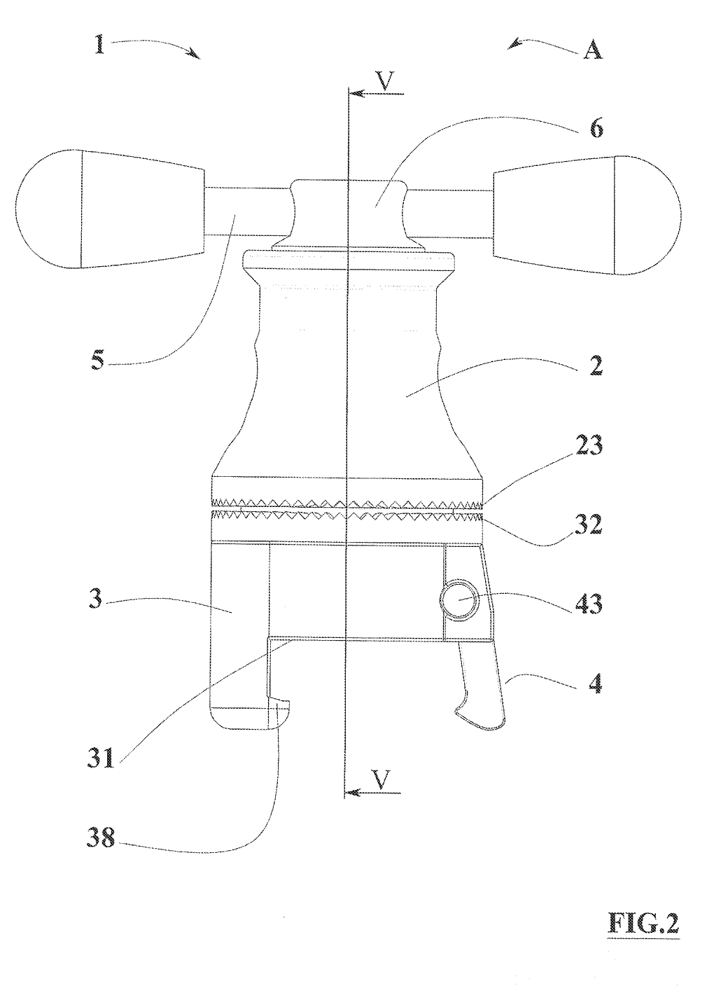

FIG. 2 shows a side view of the device according to the present invention alone;

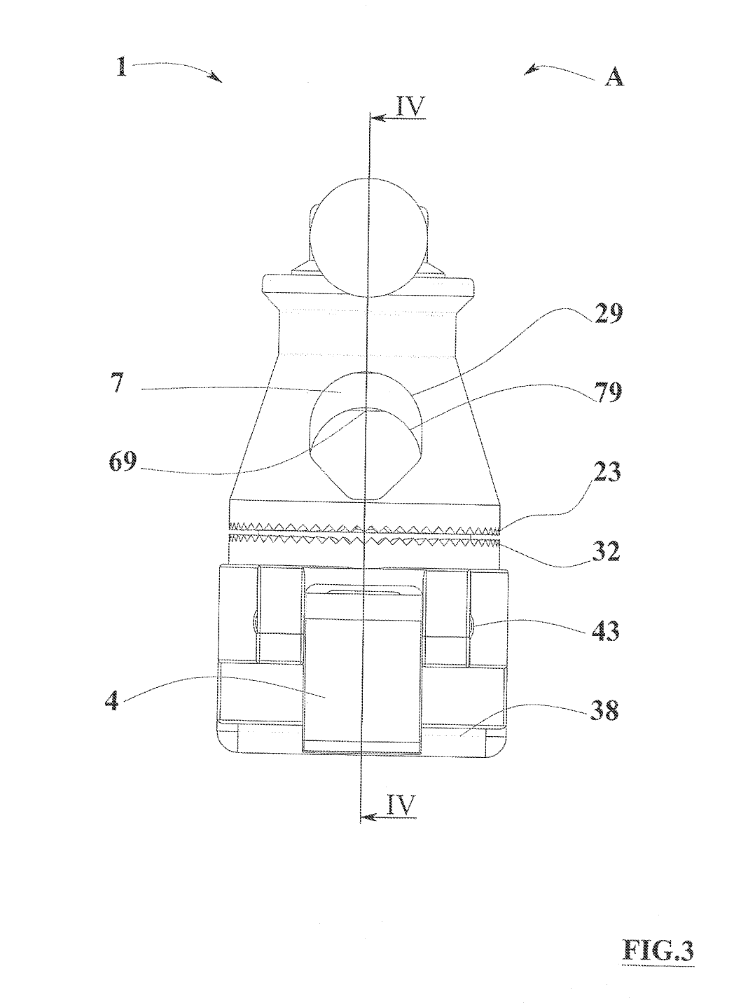

FIG. 3 shows a front view of the device shown in FIG. 2;

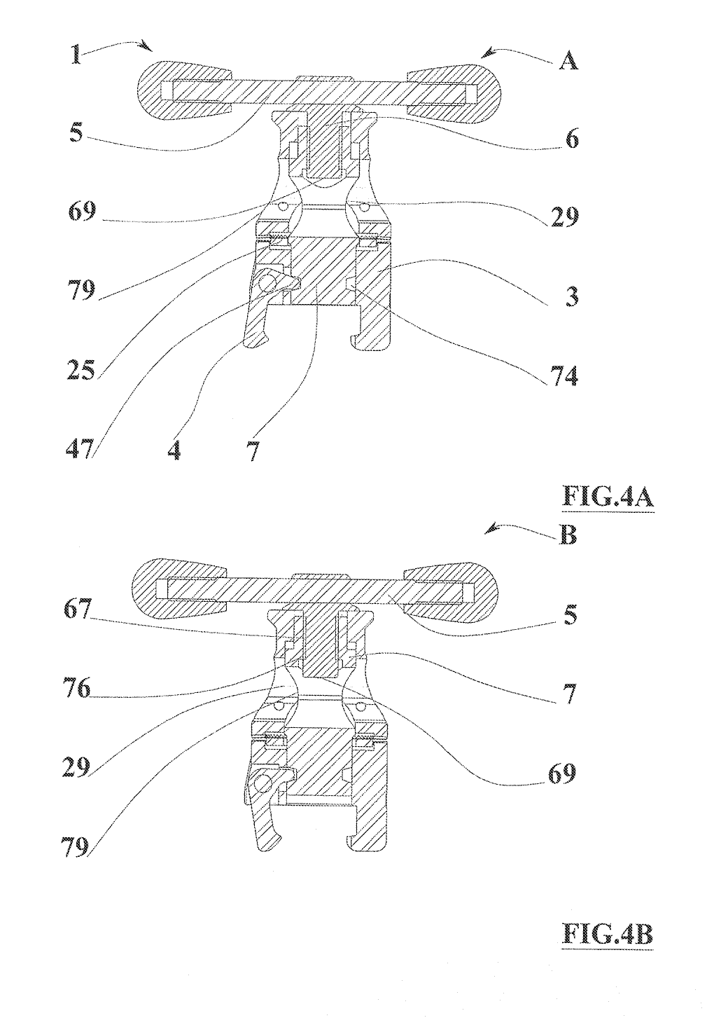

FIG. 4A shows a cross-section side view according to plane IV-IV in FIG. 3 in a release condition A;

FIG. 4B shows a cross-section side view according to plane IV-IV in FIG. 3 in a guide locked condition B;

FIG. 4C shows a partial cross-section side view of the device shown in FIG. 3 according to plane IV-IV in a third condition;

FIG. 5 shows a partial cross-section view according to plane V-V in FIG. 2;

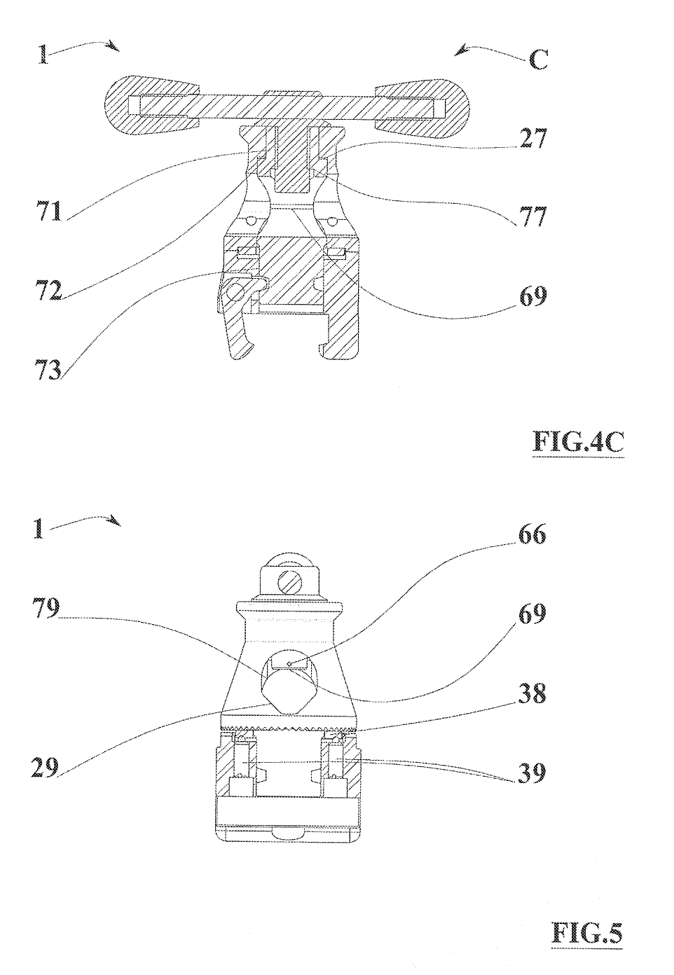

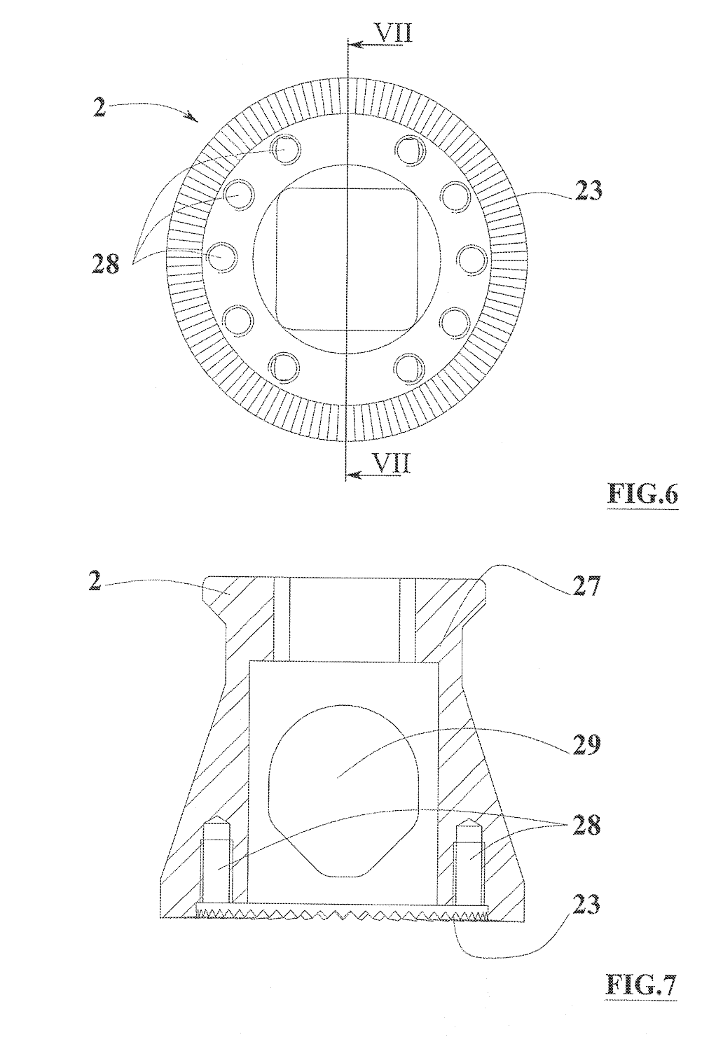

FIG. 6 shows a bottom view of a first element of the device shown in FIG. 2;

FIG. 7 shows a cross-section view according to plane VII-VII in FIG. 6;

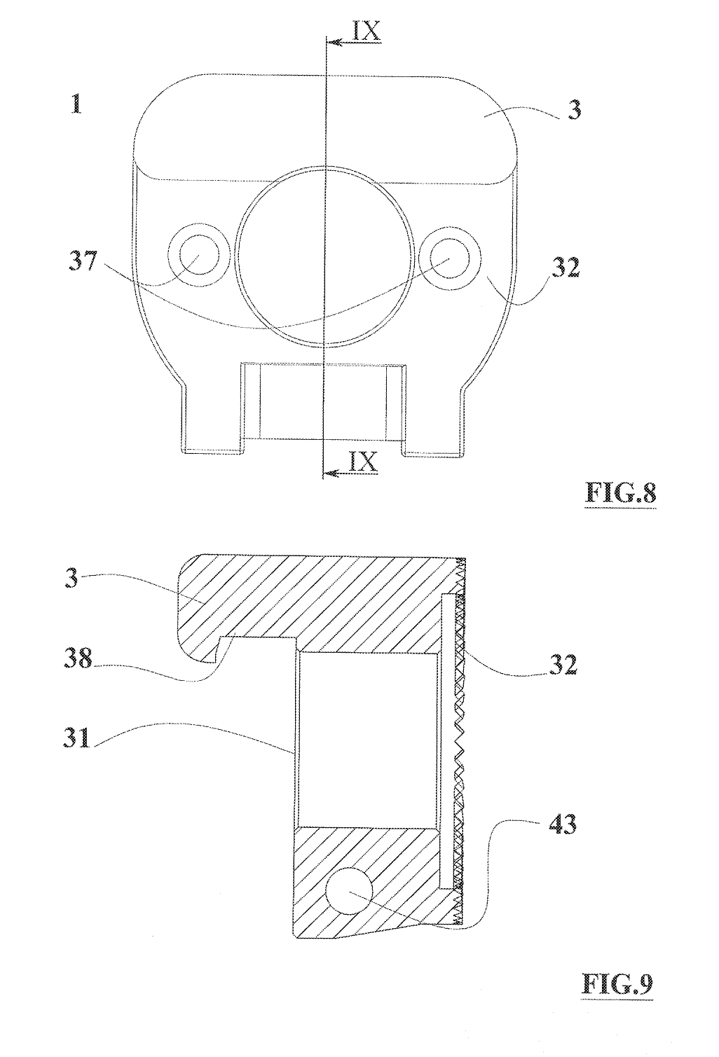

FIG. 8 shows a bottom view of a second element of the device shown in FIG. 2;

FIG. 9 shows a cross-section view according to plane IX-IX in FIG. 8;

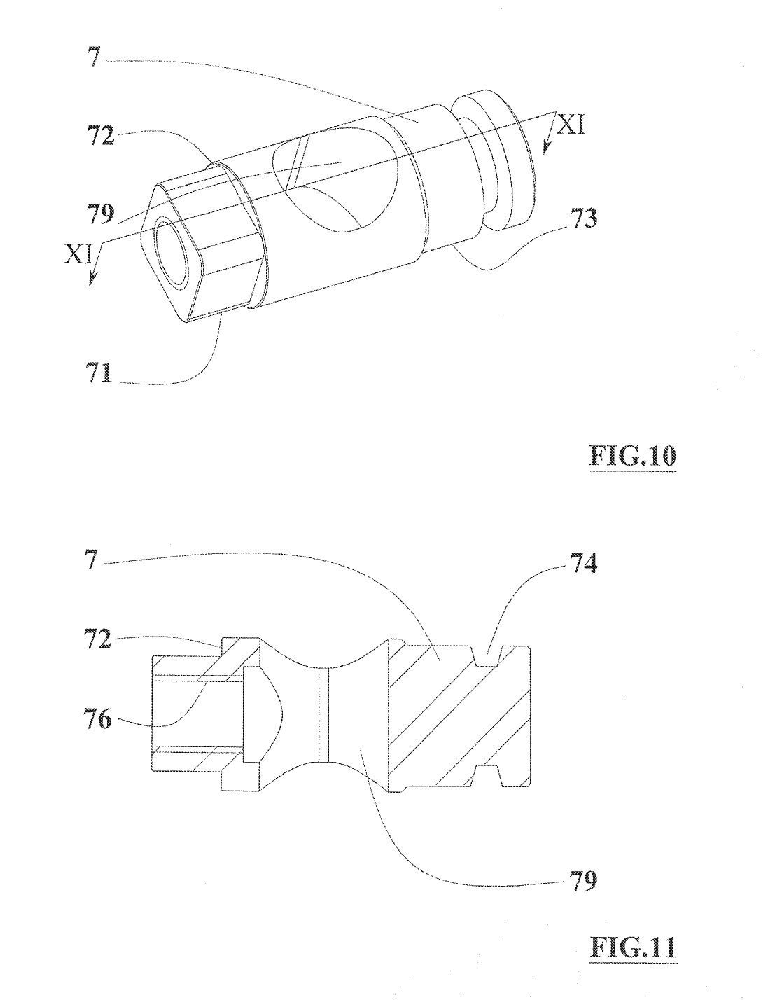

FIG. 10 shows an axonometric view of a third element of the device shown in FIG. 2;

FIG. 11 shows a cross-section view according to plane XI-XI in FIG. 10;

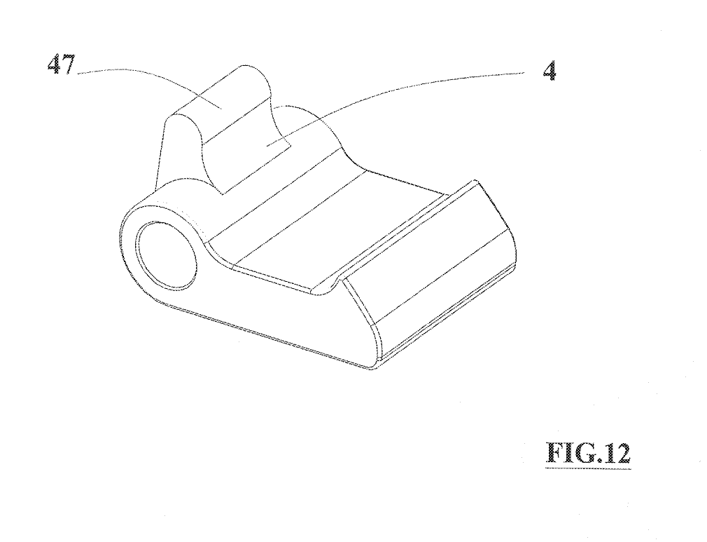

FIG. 12 shows an axonometric view of a fourth element of the device shown in FIG. 2.

With reference to FIGS. 1 thru 12, the numeral 1 identifies the fast locking device for at least one tool 9 on at least one guide element 8 according to the present invention.

In the preferred embodiment, the device 1 is used to fix tools having at least one elongated element featuring a standardized circular cross-section to guide elements featuring a standardized rectangular cross-section present at the sides of operating tables.

The device 1 comprises a fixing element 2 whose external shape is a truncated cone, provided with at least one first opening 29, which allows a tool to pass through, and diametrically crosses the fixing element 2 in correspondence with the tapered section of the truncated cone and develops parallel to the base surface of the truncated cone. The fixing element 2 also presents a variable-section transition which joins the two faces of the truncated cone.

A base 3 is removably lockable to the fixing element 2, by means of clutch means, in correspondence with the base of the truncated cone and is provided with a stop surface 31 opposite to the surface the fixing element 2 is lockable to. At an end of the stop surface 31 there is an “L”-shaped locking element 38 the function of which is to partially engage the guide element 8.

The clutch means comprise a first locking surface 23 on the fixing element 2 and a second locking surface 32 on the base 3 both featuring a sequence of tooth-like projections featuring a radial development.

A hook-like element 4 is revolvingly locked to the base 3 via a pin 43 located at the end of the stop surface 31 opposite to that where the locking element 38 is located.

The hook-like element features a projection 47 close to the pin 43 and facing the inside of the device 1.

The hook-like element 4 is movable between a release position A, in which it forms an obtuse angle with the base surface, and a locked position B, in which it forms an acute angle with the base surface and in conjunction with the locking element 38 locks the guide element 8 against the base 3.

A cursor element 7 is coaxially sliding internally to the fixing element 2 and to the base 3 and is provided with a second opening 79, which is substantially in line with the first opening 29 of the fixing element 2 in an operating condition, and a hollow 74 which engages the projection 47 of the hook-like element 4 so as to make it rotate around the pin 43 whenever the cursor element 7 moves internally to the base 3 and the fixing element 2.

The cursor element 7 features a circular section 73 close to a portion thereof sliding inside the base 3, to make it possible a mutual rotation between the cursor element 7 and the base 3, and a section featuring a sequence of flat faces 71 joined to each other by curved surfaces in correspondence with a portion thereof sliding inside the fixing means 3, so as to prevent a mutual rotation thereof. The circular section 73 and the flat-faces section 71 are jointed to each other by a shoulder 72 which abuts a thrust surface 27 internal to the fixing element 2 and parallel to the first locking surface 23.

A locking means 6 internally engages the cursor element 7 and is provided with an inner end 69 that leads to inside the first opening 29 and the second opening 79 to abut the tool 9 whenever the latter is located inside them.

The locking means 6 is an at least partially threaded element 67 which engages a nut screw 76 inside the cursor element 7 and close to the outer free end it is equipped with a control element 5, consisting of a sliding rocker arm or an end lever.

Also, the locking means 6 comprises, close to the inner end 69, a safety element 66 which engages a corresponding seat 77 present in the cursor element 7 to prevent a complete slip off thereof.

A number of elastic elements 39, consisting of helical springs, are interposed between the base 3 and the fixing element 2 so as to counter their mutual approach. In order to improve the operation of said elastic element 39, an annular shim 25 is interposed between the fixing element 2 and the base 3 and constitutes the thrust surface which the elastic elements 39, possibly provided with a sphere-like end to improve its smoothness, act onto.

In order to make it possible to fix tools having different cross-sections, the first opening 29 and the second opening 79 feature a tapered shape.

In a first variant of the preferred embodiment of the device 1, the fixing element 2 presents a plurality of threaded holes 28 that can be aligned to their respective holes 37 present on the base 3 and whose function is to removably lock the position between the fixing element 2 and the base element 3 at predetermined angles of rotation.

In order to make it easier the identification of the correct angle of rotation on the outer surfaces of the fixing element 2 and of the base 3, there are reference indicators which show the angle of rotation with respect to an initial position.

In a second variant of the preferred embodiment of the device 1, the locking means 5 is provided with audible, tactile, or visual indicators to indicate the different operating conditions of the device 1 to the operator.

The device 1 supports the following operating conditions:
release condition A, illustrated in FIG. 4A, in which the device 1 is not locked to the guide element 8 nor does it lock the tool 9, and the hook-like means 4 features an obtuse angle with respect to the stop surface 31;
guide locked condition B, illustrated in FIG. 4B, in which the inner end 69 of the locking means 6 abuts the tool 9, and exerts a limited pressure onto the latter, and makes the cursor element 7 move and drives in its own movement the hook-like element from the release condition A to the locked position B in which it approaches the guide element 8 and locks it against the base 3, in this condition the fixing element 2 being able to rotate with respect to the base 3 and the tool 9 being able to move;
rotation locking condition C, illustrated in FIG. 4C, in which because of the increased pressure exerted by the locking means 6 onto the tool 9, the cursor element 7 overcomes the resistance offered by the elastic element 38 and makes the fixing element 2 approach the base 3 up to mutually locking them, in such condition the tool 9 being free to move, such movement being however partially countered by the elastic element 38;
tool locking condition, illustrated in FIG. 1, in which the locking means 7 is squeezed and the inner end 69 exerts such a force onto the tool 9 as to lock it.

In the operation of the preferred embodiment, the operator shall place the device 1, set to its release condition A, onto the guide 8 it shall be fixed to and makes it move along the latter up to the correct position. Having reached the correct position, the operator shall insert the tool 9 into the first opening 29 and the second opening 79, advantage being taken of the resistance offered by the elastic element 39 which limits the sliding capabilities of the tool.

Then the operator shall operate the locking means 6 by making the control element 5 rotate by approximately ¼ of turn clockwise, so as to make the cursor element 7 move, hence the hook-like element 4 rotate towards the base 3 up to locking the guide element 8 in correspondence with the guide locked conditions B.

In the guide locked condition B, the operator can vary the angle of rotation between the base 3 and the fixing element 2 at will to correctly position the tool as a function of the specific requirements of every operation.

Having reached the desired angle, the operator shall turn the control element 5 by a further ¼ of turn clockwise, which makes the cursor element 7 overcome the resistance of the elastic elements 38 and move further and approach the base 3 to the fixing element 2 up to locking them in the desired position, in correspondence with the rotation locking condition C.

In this condition the operator can easily move the tool 9 with respect to the device 1 up to reaching the optimum position for the operation.

Finally the operator shall turn the control element 5 by a further ¼ of turn clockwise to lock the tool 9, in correspondence with the tool locked condition.

Should it be necessary to vary the position of the tool 9 with respect to the device 1, the operator shall turn the control element 5 by approximately ¼ of turn counterclockwise so as to relieve the pressure of the locking means 6 onto the tool 9 and to make its movement possible.

Similar considerations apply, should it be necessary to vary the rotation between the base means 3 and the fixing means 2.

The main advantage of the present invention consists in that it provides a fast locking device for at least one tool that allows to fix said device onto a support guide, to adjust the position of the device and of the tool locked thereto by operating by one hand onto one adjustment element so as to make its use simpler.

A further advantage is in that it provides a device that features a limited number of movable elements so as to reduce the implementation costs and the maintenance costs as well as to reduce its possible malfunctions.

Another advantage is in that it provides a device that offers greater possibilities of use and a greater number of adjustment facilities as referred to the locking devices known so far, while retaining a structure that is compact and doesn't hamper the operations taking place in its vicinity, if any.

1. A fast locking device for at least one tool onto at least one guide element suitable for adjusting the position of said device and at least one tool by one hand onto one adjustment element comprising:
a fixing element provided with at least one first opening for the through-passage of at least one tool;
a base removably lockable to the fixing element, via clutch means, provided with a stop surface, opposite to the fixing element, at an end of which there is a locking element to at least partially engage the guide element;
a hook-like element (4) revolvingly locked to the base between a release condition (A) and a locking condition (B) for the guide element;
a cursor element sliding internally to the fixing element and to the base and provided with at least one second opening and one hollow which engages a projection present on the hook-like element;
a locking means which internally engages the cursor element and is provided with an inner end that leads to the inside of the first opening and the second opening and abuts the tool;
at least one elastic element interposed between the base and the fixing element which counters their mutual approach;
said device being characterized in that it supports:
a release condition (A) in which the device is free from the guide element and from the tool;
a guide locked condition (B) in which the base is locked to the guide by means of a first rotation of the locking means;
a rotation locked condition (C) in which the rotation between the fixing element and the base is prevented by means of a second rotation in the same direction as the first one of the locking element;
a tool locked condition in which the tool is locked to the fixing element by means of a third rotation, in the same direction as the previous rotations of the locking element.

2. A device according to claim 1, wherein the cursor element features a circular section portion slidable internally to said base and a flat-faces section portion slidable internally to the fixing means, said sections being joined by a shoulder which abuts a thrust surface internal to the fixing element.

3. A device according to claim 1, wherein the locking means comprises, close to the inner end, a safety element which engages a corresponding seat present in the cursor element to prevent the complete slip off thereof.

4. A device according to claim 1, wherein the fixing element presents a plurality of threaded holes aligned to their respective holes of the base to lock the fixing element to the base at predetermined angles of rotation.

5. A device according to claim 1, wherein the outer surfaces of the fixing element and of the base comprise references which show the angle of rotation as referred to an initial position.

6. A device according to claim 1, wherein the first opening and the second opening feature a tapered shape to receive tools having different transversal sections.

7. A device according to claim 1, wherein the locking means is provided with audible, tactile, or visual indicators alternatively to indicate the different operating conditions of the device.

8. A device according to claim 1, wherein the device further comprises, between the fixing element and the base, an annular shim which at least one elastic element acts onto.
